(19) United States
(12) Patent Application Publication
(10) Pub. No.: US 2025/0101491 A1
(43) Pub. Date: Mar. 27, 2025

(54) PHAGE MEDIATED IMMUNO-PCR FOR THE DIAGNOSIS OF ALZHEIMER'S DISEASE

(71) Applicant: UNIVERSITA’ DEGLI STUDI DI MESSINA, MESSINA (ME) (IT)

(72) Inventors: Salvatore GUGLIELMINO, MESSINA (ME) (IT); Maria Giovanna RIZZO, MESSINA (ME) (IT); Domenico FRANCO, MESSINA (ME) (IT); Laura Maria DE PLANO, MESSINA (ME) (IT); Sabrina CONOCI, MESSINA (ME) (IT); Salvatore CUZZOCREA, MESSINA (ME) (IT); Emanuela ESPOSITO, MESSINA (ME) (IT); Irene PATERNITI, MESSINA (ME) (IT); Michela CAMPOLO, MESSINA (ME) (IT)

(73) Assignee: UNIVERSITA’ DEGLI STUDI DI MESSINA, MESSINA (ME) (IT)

(21) Appl. No.: 18/833,257

(22) PCT Filed: Jan. 27, 2023
(86) PCT No.: PCT/IB2023/050713

§ 371 Date: Jul. 25, 2024

(30) Foreign Application Priority Data
Jan. 27, 2022 (IT) 102022000001400

(51) Int. Cl. C12Q 1/6804; C12N 15/10; C12Q 1/6806; C12Q 1/686; G01N 33/68

(52) U.S. Cl. CPC C12Q 1/6804; C12N 15/1037; C12Q 1/6806; C12Q 1/686; G01N 33/6854; G01N 33/6896; G01N 2333/4709; G01N 2800/2821

(57) ABSTRACT

Method for diagnosing Alzheimer's disease in an individual, comprising the steps of providing at least one serum sample derived from the individual, providing at least one preparation of phage clones expressing peptides that mimic conformational epitopes of the Aβ-42 peptide, reacting said at least one serum sample with said at least one preparation of phage clones expressing peptides that mimic conformational epitopes of Aβ-42, so that antibodies that may be present in said serum and are directed against the Aβ-42 peptide bind to the peptides expressed by phage clones of said preparation of phage clones, detecting, by real-time PCR technique, the quantity of phage clones of said preparation to which said antibodies have bound, and determining, based on the quantity of phage clones of said preparation to which said antibodies have bound, whether the individual from whom said serum sample was derived suffers from Alzheimer's disease.

### TECHNICAL FIELD

The present invention relates to a method for diagnosing Alzheimer's disease and determining the different pathological stages of such disease.

### BACKGROUND ART

Alzheimer's Disease (AD) is the most common neurodegenerative disease, representing approximately two-thirds of all the cases of dementia, and affects up to 20% of individuals older than 80 years. This disease causes a progressive and irreversible decline in memory as well as an impairment of various other cognitive abilities. The disease is characterized by the destruction of nerve cells and neural connections in the cerebral cortex of the brain and a significant loss of brain mass.

There are several processes involved in Alzheimer's disease, including the formation of neurofibrillary plaques in the spaces between nerve cells, and of neurofibrillary tangles inside said cells. Such plaques consist of β-amyloid protein (Aβ), including the Aβ-42 peptide, which is reported to be toxic to neurons, causing inflammation or increasing the production of free radicals. Tangles, instead, consist of chemically altered tau proteins.

At present, the diagnosis of Alzheimer's disease is based on the analysis of a patient's clinical history as well as on neurological exams and neuropsychological tests. Neuropsychological tests, including, for example, the Mini-Mental State Examination (MMSE), are widely used to assess changes in a patient's cognitive abilities. Furthermore, the single-photon emission computed tomography (SPECT) and the positron emission tomography (PET) can be very useful in association with the assessments of the mental state. However, a confirmed diagnosis of Alzheimer's disease can be obtained only through post-mortem identification of neurofibrillary tangles and/or abnormal deposits of β-amyloid plaques in the brain.

Several studies focused on providing non-invasive diagnostic methods, based on the presence of biomarkers in several body fluids, such as saliva, urine and blood. In particular, the serum levels of antibodies specifically binding the β-amyloid peptide (Aβ-autoantibodies) in subjects suffering from Alzheimer's disease and in control subjects not suffering from said disease were intensively studied as potential biomarkers. However, the results of these studies appeared inconsistent due to several factors affecting the detection of G immunoglobulins (IgG) specific against Aβ-42, including non-specific bindings, low avidity of the antibodies in serum, wrong diagnoses, circulation of Aβ-autoantibodies, both free and bound to the Aβ-42 antigen and, not least, the structural conformation of Aβ-42.

The object of the present invention is to overcome the limitations of prior art by providing a method for diagnosing Alzheimer's disease that is non-invasive, reliable and fast.

A further object of the present invention is to provide a method that is also capable of diagnosing the different pathological stages of Alzheimer's disease.

These and other objects are achieved with the method for diagnosing Alzheimer's disease as claimed in the appended claims.

### SUMMARY OF INVENTION

The method for diagnosing Alzheimer's disease in an individual according to the present invention comprises the steps of:
providing at least one serum sample derived from the individual,
providing at least one preparation of phage clones expressing peptides that mimic conformational epitopes of the fibrillated Aβ-42 peptide, i.e., peptides that act as mimotopes of fibrillated Aβ-42,
reacting said at least one serum sample with said at least one preparation of phage clones expressing peptides that mimic conformational epitopes of fibrillated Aβ-42, so that antibodies that may be present in said serum and are directed against the fibrillated Aβ-42 peptide bind to the peptides expressed by the phage clones of said preparation of phage clones,
detecting, by real-time PCR technique, the quantity of phage clones of said preparation to which said antibodies have bound, and
determining, based on said quantity of phage clones to which said antibodies have bound, whether the individual from whom said serum sample was derived suffers from Alzheimer's disease.

Preferably, the method of diagnosis further comprises the step of determining, based on the quantity of phage clones to which said antibodies have bound, the pathological stage of Alzheimer's disease of the individual from which said serum sample was derived, said pathological stage being distinguished as either severe dementia or moderated dementia or mild dementia.

According to the present invention, the phage clones expressing peptides that mimic conformational epitopes of Aβ-42 are included in the group consisting of:
phage clones 12CIII1 expressing peptides with pattern GGGCIEGPCLEG,
phage clones 12III1 expressing peptides with pattern RWPPHFEWHFDD,
phage clones 12III15 expressing peptides with pattern WEYDRYRGWHIG,
phage clones 12IV14 expressing peptides with pattern GGHWEWHADYNL,
phage clones 12CIII3 expressing peptides with pattern WVGCHGEWCGVW, e
phage clones 12CIII4 expressing peptides with pattern HRGCIEGPCLDA.

According to the present invention, the method carried out by using preparations of phage clones 12CIII1 or 12CIII4 allows to determine whether the individual suffers from Alzheimer's disease, the method carried out by using preparations of phage clones 12III1,12III15 and 12IV14 allows to determine whether the individual suffers from Alzheimer's diseases and how severe the disease is (pathological state), and the method carried out by using preparations of phage clones 12CIII3 allows to determine the severity (pathological stage) of Alzheimer's disease.

Preferably, the method of diagnosis uses preparations of each of said phage clones, or only preparations of 12CIII1 and 12CIII3.

The method of diagnosis further comprises the step of providing a negative control based on a preparation of control phage clones expressing peptides that do not mimic conformational epitopes of the Aβ-42 peptide. The phage clones expressing peptides that do non mimic conformational epitopes of Aβ-42 are preferably phage clones 9IV1.

According to the invention, the step of detecting, by real-time PCR technique, the quantity of phage clones of said preparation to which said antibodies have bound preferably provides, prior to carrying out the real-time PCR technique, for removing the phage clones to which said antibodies have not bound and releasing, by thermal lysis, the phage DNA of the phage clones to which said antibodies have bound.

According to an embodiment of the invention, the quantity of phage clones of the preparation to which the antibodies have bound is preferably determined in terms of Cycle threshold (Ct) obtained by means of real-time PCR technique.

According to a further embodiment of the invention, the quantity of phage clones of the preparation to which the antibodies have bound is detected by measuring the weight of the bound phage clones after carrying out the PCR real-time technique.

### BRIEF DESCRIPTION OF DRAWINGS

These and other features and advantages of the present invention will become evident from the following description of preferred embodiments given by way of non-limiting example with reference to the annexed drawings, in which parts identified with identical or similar reference numerals denote parts having identical or similar function and construction, and in which:

FIG. 1 schematically shows a microtitration plate used in the method of diagnosis according to the present invention, in which the phage clones present in the respective wells are indicated;

FIG. 2 schematically shows the principles of the method in which antibodies present in a serum sample from an individual suffering from Alzheimer's disease are immobilized on a surface functionalized with (a) the G protein and (b) a phage clone bound to one of said antibodies;

FIG. 3 shows the standard calibration curve obtained by means of real-time PCR and used in the method of diagnosis according to the present invention;

FIGS. 4a-4d show, for four different serum samples, the diagrams of the percentages of phage clones bound to the antibodies, said percentages being obtained with the method of diagnosis according to the present invention;

FIGS. 5a-5f show the ROC curves of the diagnostic tests carried out with the phage clones for serum samples from individuals suffering from Alzheimer's disease.

### DESCRIPTION OF EMBODIMENTS

A method for diagnosing the pathological stages of Alzheimer's disease in an individual is described below, said method being based on the use of phage clones expressing peptides that mimic the conformational epitopes of the Aβ-42 peptide as well as on the detection of quantities of such phage clones bound to anti-Aβ-42 antibodies that may be present in the individual's serum.

According to the present invention, the phage clones used in the method of diagnosis were selected by means of the known phage display technique, in a process referred to as “double binding”, this process, too, being known (see International Patent Application No. PCT/IB2017/057422), identifying phage clones capable of binding G immunoglobulins (IgG) of subjects suffering from Alzheimer's disease and monoclonal antibodies directed against the capsule antigen Caf1. In fact, the Applicant carried out researches, through bioinformatic tools, for identifying proteins having conformational patterns homologous to the Aβ-42 peptide, and such researches showed that, among microbial proteins, the epitope region of the F1 capsule antigen (Caf1) synthetized by the bacterium Yersinia pestis exhibits significant structural homology to the fibrillary form of Aβ peptides.

The selected phage clones thus express, as mentioned above, peptides that mimic conformational epitopes of the Aβ-42 peptide, i.e., are mimotopes of Aβ-42, and can therefore be recognized by the antibodies present in the serum of an individual suffering from Alzheimer's disease. The reactivity of such isolated phage clones was assessed, ascertaining that a significant level of IgGs in subjects suffering from Alzheimer's disease binds to such phage clones, thus allowing to distinguish these subjects from subjects that do non suffer from said disease.

The phage clones selected by means of the above-mentioned techniques and used in the method of diagnosis according to the present invention belong to the group of phage clones consisting of: 12CIII1, 12III1, 12III15, 12IV14, 12CIII3, 12CIII4.

The pattern of the peptides expressed by the above-mentioned phage clones is shown in Table 1.

TABLE 1

| Phage clones | Peptides (pattern) |
|---|---|
| 12CIII1 | GGGCIEGPCLEG |
| 12III1 | RWPPHFEWHFDD |
| 12III15 | WEYDRYRGWHIG |
| 12IV14 | GGHWEWHADYNL |
| 12CIII3 | WVGCHGEWCGVW |
| 12CIII4 | HRGCIEGPCLDA |

In particular, said selected phage clones are divided into three conformational clusters on the β-amyloid:
cluster 1: it comprises 12III1, 12III15 and 12IV14, which exhibit conformational homology along the N-terminal region of the fibrillated β-amyloid;
cluster 2: it comprises 12CIII1, which exhibits conformational homology along the inner-central part of the fibrillated β-amyloid;
cluster 3: it comprises 12CIII4 and 12CIII3, which exhibit conformational homology along the C-terminal region of the fibrillated β-amyloid.

According to the method of diagnosis according to the present invention, serum samples from an individual are reacted with respective preparations of said phage clones, in order to detect anti-Aβ-42 antibodies that may be present in the serum, thanks to the fact that such antibodies bind to the peptides expressed by the phage clones of the preparations.

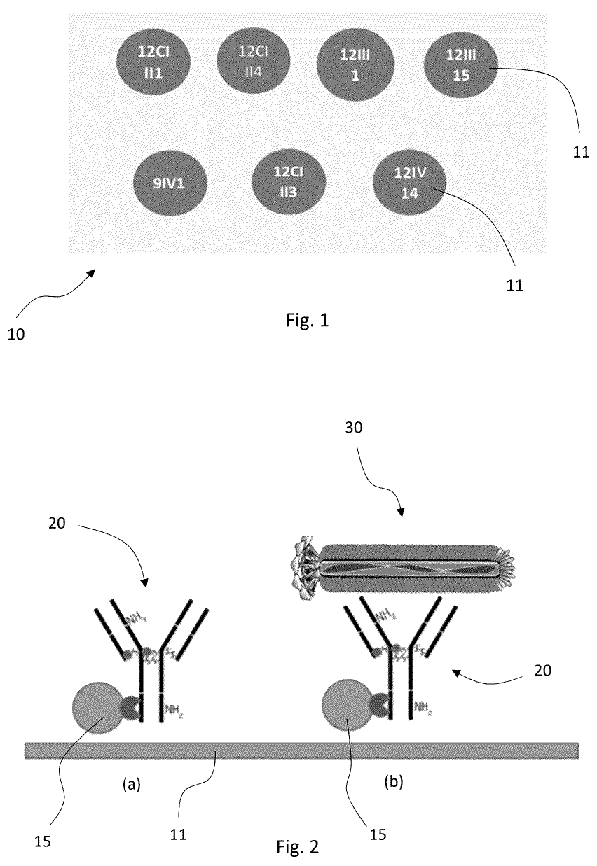

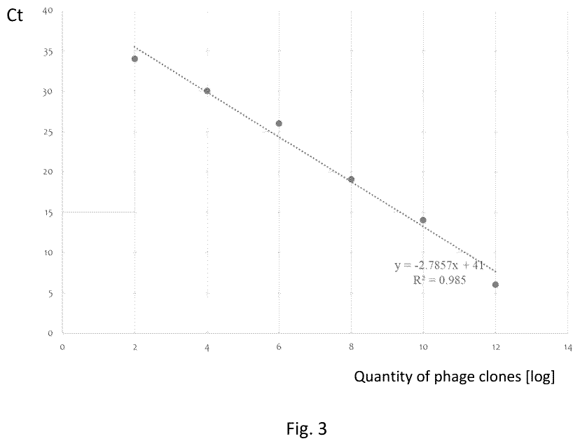

Referring to FIGS. 1 and 2, said reaction is carried out in a microtitration plate 10 (for example, the Nunc Maxisorp™ polystyrene plate), in which wells 11 are at first treated with G protein, indicated by numeral 15, the function of which is to immobilize the antibodies 20 present in the serum samples that will subsequently be added to the wells. For example, the aforesaid treatment is carried out by introducing, into each well, 200 μl of G protein (0.5 μg/ml) in a NaHCO₃/NaCO₃ buffer and incubating at 4° C. overnight. The wells of the plate are then washed, for example, with 250 μl/well of washing buffer (PBS; 0.05% Tween20). After washing, the serum samples from an individual are introduced into the wells. For example, 200 μl/well of serum with a dilution 1:50 are added. The plate is then incubated, preferably at 37° C. at 100 rpm for 1 hour. Blocking buffer is then added to the wells, for example 300 μl/well of TBS (Tris Buffered Saline) blocking buffer containing 5% (p/v) of skimmed milk (TBSM), and the plate is then incubated at 37° C. under statics for 2 hours. Washings of the wells are then carried out, for example three washings with 250 μl/well of washing buffer. Subsequently, the preparation of a respective phage clone 30 is introduced into each well, preferably at a concentration of 10¹¹ TU/mL. Incubation is then carried out, for example at 37° C. at 100 rpm for 1 hours. The phage clones 30 added to the wells are then bound to the antibodies present therein, as shown in FIG. 2.

According to the present invention, before proceeding to the detection of the quantity of phage clones to which the antibodies have bound, at first the phage clones not bound by the antibodies are removed and then the bound phage clones are thermally lysed. For example, such removal of unbound phage clones is performed by adding TBSET (TBS buffer containing 5 mM EDTA and 0.1% Tween-20), 50 μl of distilled water are then added to each well and the plate is incubated in bain-marie (at 95° C. for 10 minutes) to lyse the bound phages.

The method of diagnosis according to the present invention therefore provided for detecting, by means of real-time PCR technique, the quantity of phage clones left in each well, i.e., the quantity of phage clones to which the antibodies have bound, and, based thereon, determining whether the individual from which the serum sample was obtained suffers from Alzheimer's disease, as well as the pathological stage of the disease.

For the real-time PCR reaction, a PCR premix was used consisting of 4 μl of phage lysate as template, 5 μl of Taq supermix (2×) and 0.2 mM of forward primer (E24-5′GCTACCCTCGTTCCGATGCTGTC3′) and reverse primer (M13-40-5′GTTTTCCCAGTCACGAC3′).

The program of the real-time PCR steps used in the present method of diagnosis is, for example, as follows: 94° C. for 5 minutes, followed by 30 cycles at 94° C. for 30 seconds, at 52° C. for 30 seconds and at 72° C. for 30 seconds.

For the real-time PCR, a threshold level above the background signal was determined and the values of cycle thresholds (Cts) were set up as the numbers of cycles required for the measured fluorescence to intersect said threshold level. The quantity of phage clones present in a well is then detected in terms of cycle threshold (Ct) values. As is known, the measured cycle threshold (Ct) values are inversely proportional to the quantity of template DNA to be replicated, i.e., the DNA of the phage clones (i.e., the lower the cycle threshold value, the larger the quantity of phage clones present in the well).

The real-time PCR technique used in the method of diagnosis according to the present invention is thus referred to as Phage mediated Immuno-RT-PCR (PI-RTPCR).

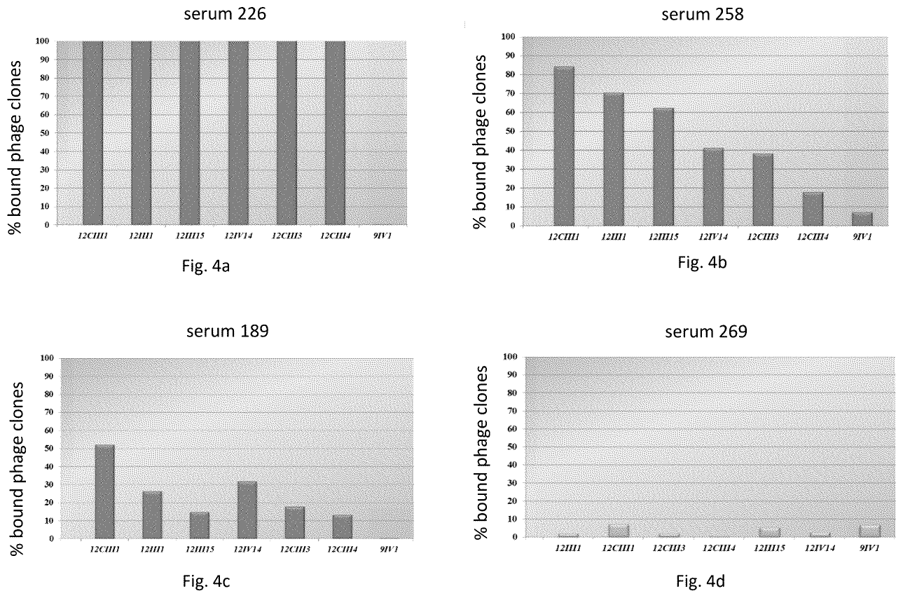

In order to obtain the quantity of phage clones corresponding to the determined cycle threshold value measured with the PI-RTPCR technique, a calibration standard curve (shown in FIG. 3) is used, obtained by measuring the cycle threshold (Ct) value for known quantities of phage clones. The known quantities of phage clones used and the corresponding cycle threshold (Ct) values detected are shown in Table 2.

TABLE 2

| Quantity of phage | Cycle threshold |
|---|---|
| clones [TU/mL] | (Ct) |
| 1 × 10² | 34 |
| 1 × 10⁴ | 30 |
| 1 × 10⁶ | 26 |
| 1 × 10⁸ | 19 |
| 1 × 10¹⁰ | 14 |
| 1 × 10¹² | 6 |

To convert a determined cycle threshold (Ct) value measured with the PI-RTPCR technique into a corresponding quantity of phage clones, the quantity of phage clones corresponding to said cycle threshold (Ct) value is extrapolated from the calibration standard curve. This is performed, for example, by using the Excel function “FORECAST.LINEAR (x, known_y's, known_x's)”, where “known_y's” and “known_x's” are the known quantities of phage clones and the corresponding known cycle threshold values, shown in the previous table, and “x” is the determined measured cycle threshold (Ct) value for which the corresponding quantity of phage clones is to be obtained, i.e., as mentioned above, the quantity of phage clones bound to the antibodies.

The quantity of phage clones bound to the antibodies is expressed as percentage according to the following formula:

% phage clone that binds to the target=(phage b/phage i)×100,
where “phage b” is the phage quantity (in mg/mL) bound to the antibodies (target), calculated as shown above, i.e., by measuring the cycle threshold (Ct) by means of the PI-RTPCR technique and comparing such value with the calibration standard curve, and “phage i” is the initial concentration (input) of phage clones (in mg/mL), determined, for example, by means of visible UV spectroscopy at 269 nm, wherein a 260 nm absorbance unit corresponds to 2.2×10¹² TU/mL.

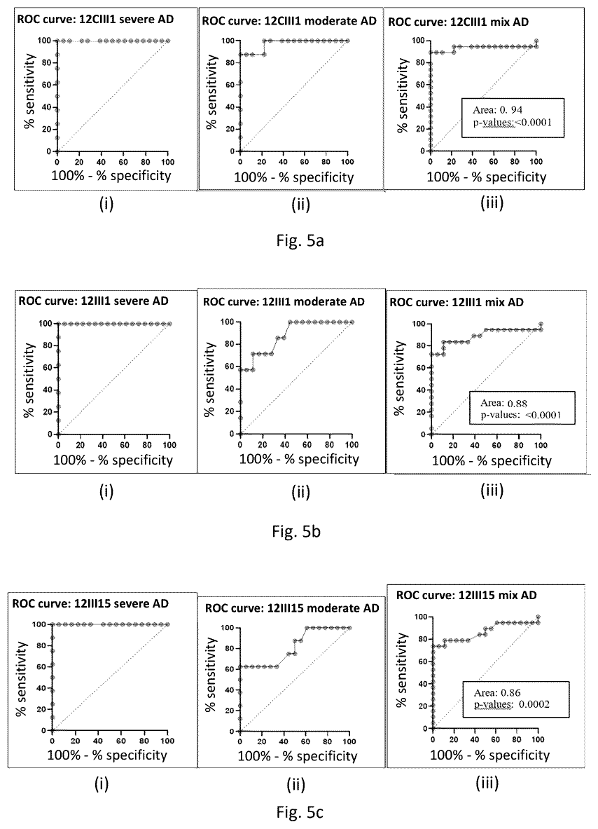

Referring to FIGS. 4a-d, the values obtained by the Applicant for the percentages of phage clones that bind to the antibodies are graphically shown for four different sera. In particular, FIG. 4a shows the values obtained for a serum (serum “226”) derived from an individual suffering from Alzheimer's disease with severe level of dementia (MMSE<10), FIG. 4b shows the values obtained for a serum (serum “258”) derived from an individual suffering from Alzheimer's disease with moderate level of dementia (10<MMSE<20), FIG. 4c shows the values obtained for a serum (serum “189”) derived from an individual suffering from Alzheimer's disease with mild level of dementia (MMSE>20), and FIG. 4d shows the values obtained for a control serum (serum “269”) derived from an individual not suffering from Alzheimer's disease. In addition, for each serum, the percentage of bound phage clones for a control phage clone, i.e., the phage clone 9IV1, expressing peptides with pattern YNTIPSRRV that are not mimotopes of Aβ-42 is also shown.

The results shown in the aforesaid diagrams indicate that for the selected phage clones the percentage binding to the antibodies of an individual suffering from Alzheimer's disease stands at 100% for forms of severe dementia and decreases to a percentage higher than 10% for forms of mild dementia. Said percent values obtained for individuals suffering from Alzheimer's disease are remarkably higher than those obtained for individuals not suffering from Alzheimer's disease, which are always lower than 10%.

The data obtained with said phage clones and sera are statistically analyzed by means of ROC (Receiver Operating Characteristic) curves, known in decision theory, which make it possible to measure the accuracy of the diagnostic test according to the invention over the entire range of possible threshold values that distinguish an individual suffering from Alzheimer's disease from an individual not suffering from said disease. As is known, “sensitivity” and “1-specificity”, represented, respectively, by the fraction of true positives (TPR, True Positive Rate), i.e., the proportion of individuals having a positive test value among all those actually suffering from the disease, and the fraction of false positives (FPR, False Positive Rate), i.e., the proportion of individuals who, though having a positive test value, do not suffer from the concerned disease among those not actually suffering from the disease, are shown along the two axes of said curves. The ROC curve thus measures the accordance between the diagnostic test and the presence/absence of the disease by evaluating the Area Under Curve (AUC): an area of 1 represents a perfectly accurate test, whereas an area of 0.5 (area under a straight line at 45° passing through the origin of the axes) represents a test with random results (null hypothesis). The ROC curve further makes it possible to identify the optimal threshold value (the so-called best cut-off), i.e., the test value maximizing the difference between true positives and false positives.

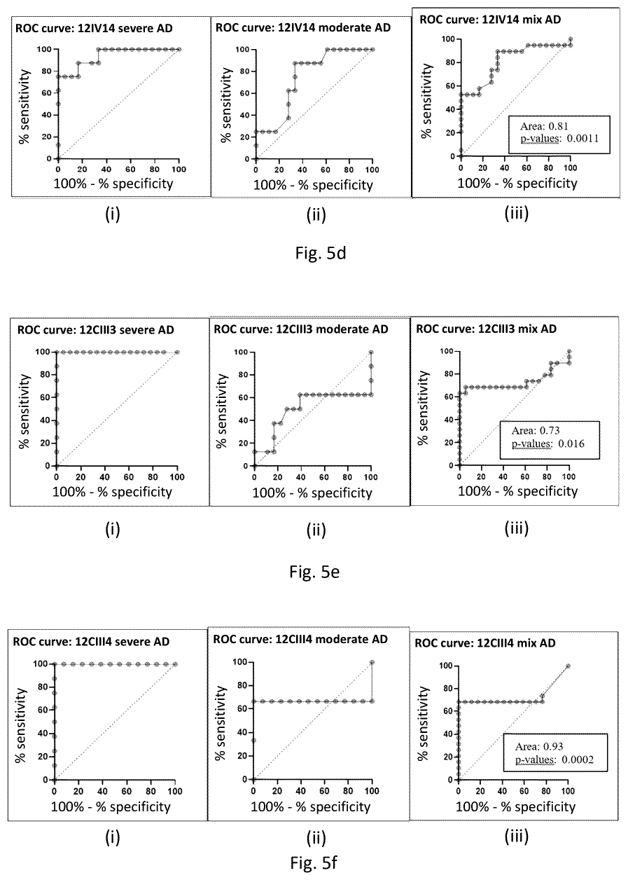

The ROC curves of the diagnostic tests performed for each of the phage clones (the selected ones expressing mimotopes of Aβ-42 as well as the control phage clone) with i) sera of individuals suffering from a severe form of Alzheimer's disease, ii) sera of individuals suffering from a moderate form of Alzheimer's disease, and iii) sera of individuals suffering from Alzheimer's disease at different stages (severe, moderate and mild) are shown in FIGS. 5a-f, respectively. In addition, for the ROC curves obtained with sera of individuals suffering from Alzheimer's disease at different stages, values of the area under the curve and p-value (i.e., the probability of obtaining the curve observed when the null hypothesis is true) are provided. It should be noted that the obtained p-values are always very low (lower than 0.05), whereby it can be said that the obtained areas under the curve are significantly different from 0.5.

The results of the diagnostic test according to the invention performed on different sera by using each of the phage clones taken into consideration are shown in FIG. 3. In particular, sera from individuals suffering from Alzheimer's disease with severe dementia (sera “226, “218” and “224”), moderate dementia (sera “42”, “182”, “220”, “147” and “258”) and mild dementia (sera “189” and “251”) and sera from individuals not suffering from Alzheimer's disease (sera “54” e “5”) were used. For each of said sera, the value of the neuropsychological test MMSE and, for each phage clone, the cycle threshold (Ct) values obtained with the method according to the present invention as well as the weight of the phage clones bound to the antibodies are reported for each of said sera.

TABLE 3

| | | 12III1 | | 12III15 | | 12IV14 | | 12IIIC1 | | 12CIII4 | | 12CIII3 | | K- 9IV1 | |
|---|---|---|---|---|---|---|---|---|---|---|---|---|---|---|---|
| | | | phage | | phage | | phage | | phage | | phage | | phage | | phage |
| | | | weight | | weight | | weight | | weight | | weight | | weight | | weight |
| sera | MMSE | Ct | [mg/ml] | Ct | [mg/ml] | Ct | [mg/ml] | Ct | [mg/ml] | Ct | [mg/ml] | Ct | [mg/ml] | Ct | [mg/ml] |
| 226 | 7 | 11 | 5.24E−3 | 11 | 5.24E−3 | 15 | 2.38E−4 | 11 | 5.24E−3 | 11 | 5.24E−3 | 11 | 5.24E−3 | 18 | 2.07E−5 |
| 218 | 8 | 11 | 5.24E−3 | 11 | 5.24E−3 | 11 | 5.24E−3 | 11 | 5.24E−3 | 12 | 2.73E−3 | 11 | 5.24E−3 | 17 | 4.67E−5 |
| 224 | 10 | 11 | 5.24E−3 | 11 | 5.24E−3 | 11 | 5.24E−3 | 10 | 8.99E−3 | 11 | 5.24E−3 | 11 | 5.24E−3 | 16 | 1.03E−4 |
| 42 | 15 | 15 | 2.38E−4 | 16 | 1.03E−4 | 16 | 1.03E−4 | 13 | 1.21E−3 | 11 | 5.24E−3 | 20 | 4.67E−6 | 25 | 1E−7 |
| 182 | 16 | 12 | 2.73E−3 | 12 | 2.73E−3 | 12 | 2.73E−3 | 11 | 5.24E−3 | 13 | 1.21E−3 | 13 | 1.21E−3 | 22 | 6.50E−7 |
| 220 | 16 | 11 | 5.24E−3 | 11 | 5.24E−3 | 11 | 5.24E−3 | 11 | 5.24E−3 | 12 | 2.73E−3 | 11 | 5.24E−3 | 16 | 1.03E−4 |
| 147 | 17 | 12 | 2.73E−3 | 12 | 2.73E−3 | 12 | 2.73E−3 | 12 | 2.73E−3 | 11 | 5.24E−3 | 17 | 4.67E−5 | 18 | 2.07E−5 |
| 258 | 18 | 11 | 5.24E−3 | 11 | 5.24E−3 | 11 | 5.24E−3 | 11 | 5.24E−3 | 13 | 1.21E−3 | 14 | 4.95E−4 | 18 | 2.07E−5 |
| 189 | 20 | 13 | 1.21E−3 | 14 | 4.95E−4 | 14 | 4.95E−4 | 12 | 2.73E−3 | 14 | 4.95E−4 | 14 | 4.95E−4 | 21 | 1.4E−6 |
| 251 | 21 | 11 | 5.24E−3 | 12 | 2.73E−3 | 12 | 2.73E−3 | 11 | 5.24E−3 | 12 | 2.73E−3 | 14 | 4.95E−4 | 20 | 4.67E−6 |
| 54 | >30 | 18 | 2.07E−5 | 17 | 4.67E−5 | 17 | 4.67E−5 | 18 | 2.07E−5 | 18 | 2.07E−5 | 18 | 2.07E−5 | 18 | 2.07E−5 |
| 5 | >30 | 17 | 4.67E−5 | 17 | 4.67E−5 | 17 | 4.67E−5 | 16 | 1.03E−4 | 17 | 4.67E−5 | 16 | 1.03E−4 | 16 | 1.03E−4 |

The results obtained with the method according to the invention not only have high capacity of distinguishing between individuals suffering from Alzheimer's disease and healthy individuals, but they also show how phage clones 12CIII1, 12III1, 12III15, 12IV14, 12CIII3 and 12CIII4 can distinguish between individuals with severe, moderate and mild pathology. For each phage clone considered, the optimal threshold values for interpreting the diagnostic test and distinguishing an ill individual from a healthy one were determined. Such threshold values, expressed in terms of cycle threshold (Ct), are as follows:
Ct≥16: negative test (i.e., individual not suffering from Alzheimer's disease),
14<Ct<16: uncertain test,
Ct≤14: positive test (i.e., individual suffering from Alzheimer's disease).

In addition, in alternative to cycle threshold (Ct) values, in order to evaluate the test results it is possible to consider the weight of bound phage clones (Wpb), such weight being inversely proportional to the obtained cycle threshold. In particular:
Wpb≤0.1 μg/mL: negative test (i.e., individual not suffering from Alzheimer's disease),
0.1 μg/mL<Wpb<0.5 μg/mL: uncertain test,
Wpb≥0.5 μg/mL: positive test (i.e., individual suffering from Alzheimer's disease).

From the data shown in Table 3 and from FIG. 5 it results that the proposed array of phage clones determines the following associations for staging the disease:
high titer antibodies present in sera and directed against the clones 12CIII1 and 12CIII4 can be considered as indexes of the presence of Alzheimer's disease;
high titer antibodies present in sera and directed against the clones 12III1, 12III15 and 12IV14 can be considered as indexes of the presence/severity of Alzheimer's disease;
antibodies present in sera and directed against the clone 12CIII3 can be considered as an index of the severity of Alzheimer's disease;
the clone 9IV1 is used as negative control.

In a preferred embodiment of the method of diagnosis according to the invention, all the phage clones mentioned above are used. In an alternative embodiment, use is made only of the phage clone 12CIII1 for detecting the presence of Alzheimer's disease, the phage clone 12CIII3 for detecting the severity of the disease, and the control phage 9IV1.

The method of diagnosis according to the present invention, by providing an indirect measurement of the levels of antibodies against conformational epitopes of Aβ-42 thus makes it possible to distinguish between healthy individuals and ill individuals and allocate an index of severity based on the state and stage of the disease.

1. A method for diagnosing Alzheimer's disease in an individual, comprising the steps of:
providing at least one serum sample derived from the individual,
providing at least one preparation of phage clones (30) expressing peptides that mimic conformational epitopes of Aβ-42 peptide,
reacting said at least one serum sample with said at least one preparation under conditions suitable for antibodies (20) present in said serum and directed against the Aβ-42 peptide, bind to the peptides expressed by the phage clones of said preparation,
detecting, by real-time polymerase chain reaction (PCR), the quantity of phage clones (30) of said preparation to which said antibodies (20) have bound, by determining Cycle threshold (Ct) obtained by said real-time PCR or by measuring bound phage clone weight (Wpb),
wherein a (Ct)≥16 or a Wpb≤0.1 μg/mL indicates that the individual does not suffer from Alzheimer's disease and a (Ct)≤14 or a Wpb≥0.5 μg/mL indicates that the individual suffers from Alzheimer's disease.

2. The method according to claim 1, further comprising the step of determining, based on said amount of phage clones to which said antibodies have bound, the pathological stage of Alzheimer's disease of the individual from which said serum sample was derived.

3. The method according to claim 1, wherein the phage clones (30) expressing peptides that mimic the conformational epitopes of Aβ-42 are selected from:
phage clones (12CIII1) expressing peptides with pattern GGGCIEGPCLEG (SEQ ID NO: 1),
phage clones (12III1) expressing peptides with pattern RWPPHFEWHFDD (SEQ ID NO: 2),
phage clones (12III15) expressing peptides with pattern WEYDRYRGWHIG (SEQ ID NO: 3),
phage clones (12IV14) expressing peptides with pattern GGHWEWHADYNL (SEQ ID NO: 4),
phage clones (12CIII3) expressing peptides with pattern WVGCHGEWCGVW (SEQ ID NO: 5), and
phage clones (12CIII4) expressing peptides with pattern HRGCIEGPCLDA (SEQ ID NO: 6).

4. The method according to claim 3, wherein preparations of
i) phage clones (12CIII1) expressing peptides of SEQ ID NO: 1 or
ii) phage clones (12CIII4) expressing peptides of SEQ ID NO: 6 determines whether the individual suffers from Alzheimer's disease, or
iii) preparations of phage clones (12III1) expressing peptides of SEQ ID NO: 2, phage clones (12III15) expressing peptides of SEQ ID NO: 3
or
iv) phage clones (12IV14) expressing peptides of SEQ ID NO: 4 identifies individuals suffering from Alzheimer's disease as well as the pathological stage of Alzheimer's disease, and, or
v) wherein using preparations of phage clones (12CIII3) expressing peptides of SEQ ID NO: 5 determines the pathological stage of Alzheimer's disease.

5. The method of claim 1, further comprising the step of providing a preparation of control phage clones expressing peptides that do not mimic conformational epitopes of the Aβ-42 peptide.

6. The method according to claim 5, wherein the phage clones expressing peptides that do not mimic conformational epitopes of the Aβ-42 peptide are 9IV1.

7. The method of claim 1, comprising, prior to the real-time PCR detection step,
i) removing phage clones not bound by said antibodies and
ii) releasing phage DNA of antibody bound phage clones by thermal lysis.


SEQUENCE LISTING (6 sequences)

SEQ ID NO: 1 - protein, 12 aa, unidentified
  source 1..12: note = bacteriophage
GGGCIEGPCLEG

SEQ ID NO: 2 - protein, 12 aa, unidentified
  source 1..12: note = bacteriophage
RWPPHFEWHFDD

SEQ ID NO: 3 - protein, 12 aa, unidentified
  source 1..12: note = bacteriophage
WEYDRYRGWHIG

SEQ ID NO: 4 - protein, 12 aa, unidentified
  source 1..12: note = bacteriophage
GGHWEWHADYNL

SEQ ID NO: 5 - protein, 12 aa, unidentified
  source 1..12: note = bacteriophage
WVGCHGEWCGVW

SEQ ID NO: 6 - protein, 12 aa, unidentified
  source 1..12: note = bacteriophage
HRGCIEGPCLDA